(19) United States
(12) Patent Application Publication
(10) Pub. No.: US 2020/0395105 A1
(43) Pub. Date: Dec. 17, 2020

(54) INTELLIGENT HEALTH PROVIDER MONITORING WITH DE-IDENTIFICATION

(71) Applicant: Artsight, Inc. d/b/a Whiteboard Coordinator, Inc., Northfield, IL (US)

(72) Inventors: Timothy Koby, Waltham, WA (US); Andrew Gostine, Northfield, IL (US)

(73) Assignee: Artsight, Inc. d/b/a Whiteboard Coordinator, Inc., Northfield, IL (US)

(21) Appl. No.: 16/819,133

(22) Filed: Mar. 15, 2020

Related U.S. Application Data
(60) Provisional application No. 62/862,026, filed on Jun. 15, 2019.

(51) Int. Cl. G16H 10/60; G16H 40/00; G06K 9/00

(52) U.S. Cl. CPC G16H 10/60; G06K 9/00744; G06K 9/00664; G16H 40/00

(57) ABSTRACT

A system monitors a health facility while de-identifying certain objects, such as individuals and patient information, people and sensitive information. A platform associates de-identified sensor data with electronic medical data (EMD). Data captured by one or more intelligent sensors can be de-identified and used to detect events within the health facility and associated with an EMD record. For example, data from the one or more intelligent sensors can be used to detect progress of a procedure being performed on a patient having an EMD record associated with the health facility. As events during the procedure are detected based on processing of the intelligent sensor data, actions may be taken to further facilitate the procedure or optimize an outcome in a safe and efficient manner.

### CROSS REFERENCE TO RELATED APPLICATIONS

This application claims the priority benefit of provisional U.S. Patent Application Ser. No. 62/862,026, titled “Intelligent Health Provider Monitoring with De-Identification,” filed Jun. 15, 2019, the disclosure of which is incorporated herein by reference.

### BACKGROUND

Hospitals perform thousands of procedures on patients every day. These procedures require coordination of hospital staff, extensive inventory management, and well-trained operators in order to run smoothly. Hospitals don't have systems for automatically coordinating these procedures and inventory management. Rather, each hospital manages its own procedures and processes, and procedures and records are maintained by individuals with systems that are often unique to each hospital. Additionally, regulations such as the health insurance portability and accountability act (HIPAA) require that patient information be kept secure and confidential by hospital systems and staff, making monitoring of hospital procedures and the handling of data much more complicated. What is needed is an improved system for automatically monitoring and coordinating the varying needs of hospitals.

### SUMMARY

The present technology, roughly described, includes a platform that associates de-identified data with electronic medical data (EMD) using intelligent sensor data. Data captured by one or more intelligent sensors can be de-identified and used to detect events within a health facility and associated with an EMD record. For example, data from one or more intelligent sensors can be used to detect progress of a procedure being performed on a patient having an EMD record associated with the health facility. As events during the procedure are detected based on processing of the intelligent sensor data, actions may be taken to further facilitate the procedure or optimize and outcome in a safe and efficient manner.

The platform maintains a plurality of different firewalls in order to keep patient data safe, secure, and confidential. For example, the present system can be implemented with two firewalls—a hospital firewall and a network firewall. The sensors and de-identification process can operate within the hospital firewall, while an event engine that detects events based on captured data can operate over a network behind the network firewall. In this implementation, certain algorithms, such as for example de-identification algorithms, can be executed within the hospital firewall on “edge” compute devices, such that no data that can identify a patient or health worker (e.g., doctor, surgeon, nurse, and so on) is provided over a network to the event engine. Rather, all algorithms are run on the edge and data is de-identified at the edge of the hospital firewall before any of the data is uploaded to the cloud, for example for algorithm training. As such, the platform is flexible enough to be configured in several implementations while still maintaining patient privacy and data security in a manner that complies with healthcare and other rules and regulations, including but not limited to those set forth by the Health Insurance Portability and Accountability Act (HIPPA).

The platform of the present technology synchronizes the identified patient data to intelligent sensor data. Through the synchronization, the present system can train algorithms to monitor clinical settings for signals and enable prediction and improvement of patient outcomes. The platform can be integrated with all electronic health record systems, and can build data sets needed for developing artificial intelligence, such as machine learning-based prediction models, that improve patient outcomes.

The current platform can be used in a variety of applications based on a health facility requirements and preferences. For example, the current platform can be used in an operating room environment to determine the status of the operation procedure. The current platform can also be used as an inventory management system, for example to assist health facilities with tracking medical inventory, and to accurately and reliably assist administrators with restocking inventory as needed. The platform, consisting of intelligent sensors, de-identification systems, and an event engine that processes the intelligent sensor data and the identified data can be configured in a wide variety of ways to assist health facilities with their processes, procedures, infrastructure management, and other needs.

In some instances, a system can detect events in a health facility. The system can include an image processing application, an event engine, and a video database. The image processing application is stored and executes on a first computing machine. The image processing machine receives image data from one or more cameras and performs a de-identification process to the image data. The event engine is stored and executes on a second computing machine, and receives electronic medical data (EMD) associated with a patient. The event engine synchronizes the de-identified video streams with received electronic medical data. The video database receives the synchronized de-identified video stream and electronic medical data from the event engine. The stored and synchronized de-identified video stream and electronic medical data is used to update algorithms configured to identify objects in image.

In some instances, a method is used to detect events in a health facility. The method begins with receiving image data from one or more cameras by an image processing application, wherein the image processing application is stored and executing on a first computing machine. The image processing application performs a de-identification process to the received image data. Electronic medical data (EMD) is received by an event engine stored and executing on a second computing machine. The EMD is associated with a patient. The event engine synchronizing the de-identified video streams with received electronic medical data. The synchronized de-identified video stream and electronic medical data are received from the event engine by a video database. The stored and synchronized de-identified video stream and electronic medical data is used to update algorithms configured to identify objects in image.

### BRIEF DESCRIPTION OF FIGURES

FIG. 1 is a block diagram of a system for intelligently monitoring a health facility.

FIG. 2 is a block diagram of an event engine.

FIG. 3 is a block diagram of an event engine data flow

FIG. 4 is an exemplary method for intelligently monitoring a health facility.

FIG. 5 is an exemplary method for processing images to detect objects.

FIG. 6 is exemplary method for performing de-identification of an image.

FIG. 7 is exemplary method for performing facial de-identification.

FIG. 8 is exemplary method for storing de-identified video.

FIGS. 9A-9E illustrate the de-identification process of an image

FIG. 10 is an exemplary method for detecting events based on object data.

FIG. 11 is an exemplary method for initiating action based on an object detection.

FIG. 12 an exemplary method for intelligent tuning an algorithm.

FIG. 13 is a block diagram of a computing environment for implementing the present technology.

### DETAILED DESCRIPTION

The present technology, roughly described, includes a platform that associates de-identified data with electronic medical data (EMD) using intelligent sensor data. Data captured by one or more intelligent sensors can be de-identified and used to detect events within the health facility and associated with an EMD record. For example, data from the one or more intelligent sensors can be used to detect progress of a procedure being performed on a patient having an EMD record associated with the health facility. As events during the procedure are detected based on processing of the intelligent sensor data, actions may be taken to further facilitate the procedure in a safe and efficient manner.

The de-identified data may include images, video, and other data. An image processing application performs de-identification of the images by destructively replacing pixels of the image associated with a user face, head, body, nametags, charts, and other identifying information with black pixels. After de-identification, the image processing application provides the de-identified images to a video database. The image processing application can also identify objects within the images and video and provides data associated with the objects to an event engine. The objects may include things such as a patient bed, patient, surgeon, nurse, anesthesiologist, and other objects that can be found in a health facility.

Data from other intelligent sensors may also be de-identified. For example, an audio capture sensor may capture audio within a portion of a health facility, such as an operating room, and perform de-identification to the audio. For example, the audio capture system can modify a portion of the audio to disguise a voice tone. Additionally, the audio capture system can delete a portion of the audio that specifies identifiable information mentioned in the audio, such as a name of a patient, health care worker, or other person.

The EMD may include patient data stored, managed, and retrievable from a health data service. For example, the EMD may indicate user allergy information, surgery schedule, health history, and other data. Intelligent sensor data may include data received from a variety of intelligent sensors placed throughout a health facility. The intelligent sensors may include Bluetooth devices, RFID tags, audio capture systems, and other sensors. The intelligent sensors, such as for example Bluetooth devices and RFID tags, can be coupled to objects within the health facility and be detected as the objects move throughout the health facility. The data collected from the intelligent sensors can be used with the patient EMD record to determine the status of a patient procedure and perform actions based on the status.

The current platform can be used in a variety of applications based on a health facilities requirements and preferences. For example, the current platform can be used in an operating room environment to determine the status of the operation procedure. The current platform can also be used as an inventory management system, for example to assist health facilities with tracking medical inventory, and to accurately and reliably assist administrators with restocking inventory as needed. The platform, consisting of intelligent sensors, de-identification systems, and an event engine that processes the intelligent sensor data and the identified data can be configured in a wide variety of ways to assist health facilities with their processes, procedures, infrastructure management, and other needs.

In some instances, an implementation of the platform can be configured to monitor a health facility while de-identifying certain objects, such as individuals and patient information, people and sensitive information. Cameras record information within one or more rooms or locations of a health facility location. Video and/or images from the cameras are processed to identify objects and events of interest. The event information is provided to an event engine, and the videos are then processed to de-identify individuals, such as for example patients and health providers, and sensitive patient information. The de-identified videos are stored in a database, and optionally processed by an annotator before being used to update or modify image processing algorithms. The object information is processed to determine if the if an image object relates to an event of interest. If so, an alert and/or an action are taken in response to the object or event.

The platform maintains a plurality of different firewalls in order to keep patient data safe, secure, and confidential. For example, the present system can be implemented with two firewalls, a hospital firewall and a network firewall. The sensors and de-identification process can operate within the hospital firewall, while the event engine can operate over a network behind the network firewall. In this implementation, certain algorithms, such as for example de-identification algorithms, can be executed within the hospital firewall on “edge” compute devices, such that no data that can identify a patient or health worker (e.g., doctor, surgeon, nurse, and so on) is provided over a network to the event engine. Rather, all algorithms are run on the edge and data is de-identified at the edge of the hospital firewall before any of the data is uploaded to the cloud, for example for algorithm training. As such, the platform is flexible enough to be configured in several implementations while still maintaining patient privacy and data security in a manner that complies with healthcare and other rules and regulations, including but not limited to those set forth by the Health Insurance Portability and Accountability Act (HIPPA).

The platform of the present technology synchronizes the identified patient data to intelligent sensor data. Through the synchronization, the present system can train algorithms to monitor clinical settings for signals and able prediction and improvement of patient outcomes. The platform can be integrated with all electronic all health record systems, and can build data sets needed for developing artificial intelligence, such as machine learning-based prediction models, that improve patient outcomes.

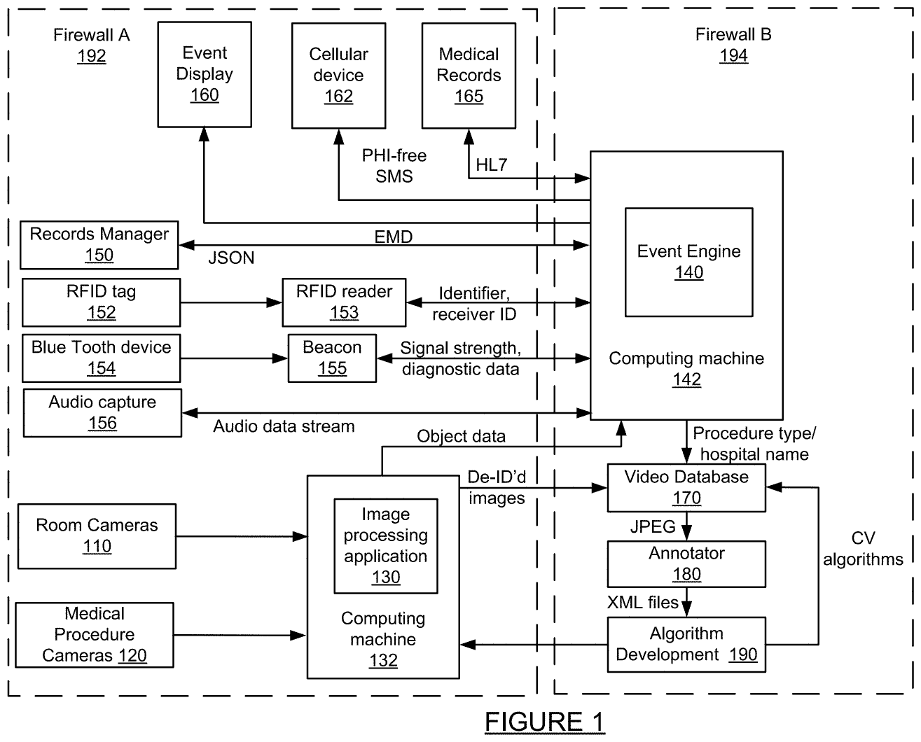

FIG. 1 is a block diagram of a system for intelligently monitoring a health facility. FIG. 1 includes cameras 110 and 120, image processing application 130 stored and executing on computing machine 132, event engine 140 stored and executing on computing machine 142, records 150, RFID device, RFID detector 153, Bluetooth device 154, beacon 155, audio capture 156, event display 160, cellular device 160, medical records 165, video database 170, annotator 180, and algorithm development 190. Components 110-165 are contained within firewall A 192. Components 150 and 170, 180, and 190 are contained within firewall B 194. Firewalls A and B may be used to prevent dissemination of information between devices and networks not included within a particular firewall. In some instances, firewall A may be implemented as a local firewall within a health provider location, and firewall B may be implemented as a network-based firewall, such as for example a Microsoft Azure firewall.

Cameras 110 and 120 capture images and/or video and provide the video to image processing application 130. Room cameras 110 may include one or more cameras positioned at different locations within a health facility. The locations may include an operating room, hallway, and other locations. Medical procedure cameras 120 may include smaller cameras such as fiber-optic cameras which capture video and/or images associated with a procedure. For example, a medical procedure camera 120 may include a laparoscopic camera. Images from cameras 110 and 120, by themselves or as part of the video, are provided to the image processing application 130.

Image processing application 130 may receive video, detect objects within images of the video, and provide object data as well as processed video to other components of the system of FIG. 1. In some instances, image processing application performs object recognition on the images to detect an object within an image. Object information for the detected object may then be provided to event engine 140. The object information may include an identifier for the camera or a location for the camera, a classifier or identifier for the object type, a confidence score, location within the image the object appears, and height and width information for the detected object. For example, object information may indicate that the image is from a particular camera associated with an operating room, the object detected is a patient, the detected object is assigned a confidence score is 0.97, the location of the center of the object within the image, and the height and length of the object within the image.

Image processing application 130 may also perform de-identification of objects and other content within an image. De-identification may include-identifying objects of interest, including patients, physicians, and other individuals, as well as identifying information, such as patient name information, physician name tags, charts, and other identifying information within the particular location. The de-identification process removes the detected information from the image. Removing information from an image may include adjusting the image so that an individual's face or identifying information is blurred, colored, or otherwise altered in a way that prevents identification of the individual or information. More details for de-identification are discussed below.

De-identified images, consisting of images that have been captured by camera 110 or 120 and de-identified by image processing application 130, are provided to video database 170. In some instances, only a subset of the captured images and videos may be provided to the video database. For example, only 10%, 8%, 5%, between 5% and 15%, or some other fraction of the total videos captured may be provided to video database 170 after being de-identified. In some instances, the videos are provided to video database 170 at times of low network activity, such as for example evening hours of a health facility.

In some instances, an image processing application 130 may exist for every location in which there is one or more cameras within a health facility. For example, if a health facility has four operating rooms, each of which have two cameras installed therein, an image processing application can be located within each of the four operating rooms having cameras. As a result, the raw video captured by cameras 110 and 120 will not leave the room in which it is captured. Rather, the raw video is processed by the local image processing application such that individuals and other patient information has been de-identified by image processing application 130, and only the video processed using de-identification is transmitted out of each operating room. Information regarding objects of interest from each image processing application within a health facility can be provided to event engine 140.

Records manager 150 may maintain health records for a patient. The records may include appointment data, laboratory and test result data, allergies, and other data typically found in a patient's medical file. Records manager 150 may provide record data to event engine 140, for example in response to a request from event engine 140. The records manager 150 may be implemented by a service provider such as “Epic” of Verona, Wis.

Radio frequency identification (RFID) tag 152 may be disposed on a physical object within a health facility. The object may be a hospital bed, crash cart, health facility badge, or some other object. When the object moves within the health facility, the RFID tag moves as well. The RFID transmits a signal when it comes into close proximity of an RFID reader. The RFID reader can transmit RFID tag data, RFID reader data, and a time at which the RFID was read by the reader. Since the location of each reader is known, the location of the RFID tag is known at the time it is read by the RFID reader.

Bluetooth device 154 may be displaced on an object within a health facility and provide a signal and diagnostic data to beacon 155. The object may be a hospital bed, crash cart, health facility badge, or some other object. When the object moves within the health facility, the Bluetooth device moves as well. The Bluetooth device 154 emits a signal, at a predetermined strength level, that is detected by beacon 155. Beacon 155 can determine the signal strength of the Bluetooth device 154, and provide the signal strength and diagnostic data to event engine 140. The diagnostic data can include the Bluetooth device ID, a power level, hours in existence, and so on.

Audio capture 156 may include one or more microphones which capture audio within a portion of the health facility, such as for example an operating room (OR). The audio capture 156 can capture the audio and provide an audio stream captured audio to event engine 140.

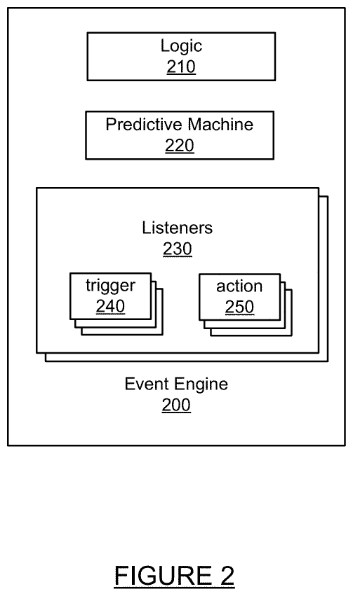

Event engine 140 receives object information from one or more image processing applications, records from record manager 150, RFID identification and receiver ID data, Bluetooth device signal strength and diagnostic data, and an audio data stream from systems 150-156. The event engine can receive and process the data, processes the received notifications to determine if a trigger has been initiated, and then performs actions as a result of the detected triggers. Event engine 140 may have a plurality of “listener” modules that can process a particular object or set of objects detected by the image processing applications. Each listener may receive one or more specified objects and determine if the received objects trigger a particular result. More detail for an event engine is discussed with respect to FIG. 2.

Video database 170 may receive de-identified images from image processing application 130 and other data, such as for example procedure type and hospital name, from event engine 140. The procedure type data identifies the type of procedure depicted in the de-identified image and the hospital name identifies the name of the hospital at which the de-identified image was captured. The de-identified image is then provided to an annotator that can detect objects in the images, for example the face of a patient in the image, and determine if the image was successfully de-identified. Additionally, the annotator can be used to identify other objects in the image, such as a patient bed. The annotated de-identified image is analyzed by algorithm development 190. Algorithm development 190 can train object detection algorithms to generate updated algorithms and then evaluate the updated algorithms. If the updated algorithms operate in an improved manner with respect to previous object detection algorithms, the updated algorithms are provided to the image processing application for use in subsequently captured images.

Event engine may publish data, event information, alerts, announcements, and other output based on the IOT data received from devices 150-156. The output may be provided to an event display 160, cellular device 160, medical records 165, or other devices suitable for communicating information. Event display 160 may be a display located in an operating room, OR prep room, or some other location within a health facility. Event display may output messages, the current state or progress of a procedure, and other information, based on processing by event engine 140. For example, if an anesthesiologist is needed in an operating room, the event engine may detect such and provide a message or update to be displayed on event display 160 regarding the need for the anesthesiologist. Similarly, event information can be transmitted to one or more individual people per their cellular device 160. In some instances, when a procedure is completed, the information can be stored in the patient's medical record, for example if event engine 140 stores data to medical records 165 (which can be the same or different from records managed by records manager 150.

FIG. 2 is a block diagram of an event engine architecture. Event engine 200 of FIG. 2 provides more detail for event engine 140 of FIG. 1. Event engine 200 includes logic 210, predictive machine 220, listeners 230, triggers 240, and actions 250. Event engine can receive data from devices and systems, such as 150-156. Logic 210 may perform an initial analysis on the data to determine if a change is detected between subsequent data. For example, logic 210 may determine if consecutive images have more than a minimum change, if consecutive audio portions have new audio or any audio, if there is any change in signal strength for a Bluetooth device reported by a beacon, and so on. If there is no change, in some instances, there may be no need to perform a more detailed analysis of the subsequent received data. If there is a change between consecutive data, then the data may be analyzed by predictive machine 220.

Predictive machine may receive data from one or more of devices 150-156 of FIG. 1, process the data, and determine if an event has occurred. In some instances, predictive machine may receive record data from records manager 150, RFID tag identifier and RFID receiver identifier data from RFID reader 153, Bluetooth device signal strength and diagnostic data from beacon 155, and audio data from audio capture device 156. Predictive machine may include one or more sets of machine learning algorithms, and the received data may serve as inputs to the machine learning algorithms. In some instances, each set of machine learning algorithm may predict the occurrence of a particular event, such as a patient entering a room, an anesthesiologist entering a room, and so on. If a particular set of machine learning algorithms provides an output indicating that the probability of the particular event happening is greater than a particular threshold (such as, for example, a threshold of 0.6), then the event may be considered to have occurred.

When an event has been detected to have occurred, the event is published to a plurality of listeners. Each listener may access the event if the listener has a “trigger” associated with the event. Hence, each listener component may listen for events that match its triggers, and each listener may perform one or more actions based on its set of one or more triggers occurring (as announced by predictive machine 220).

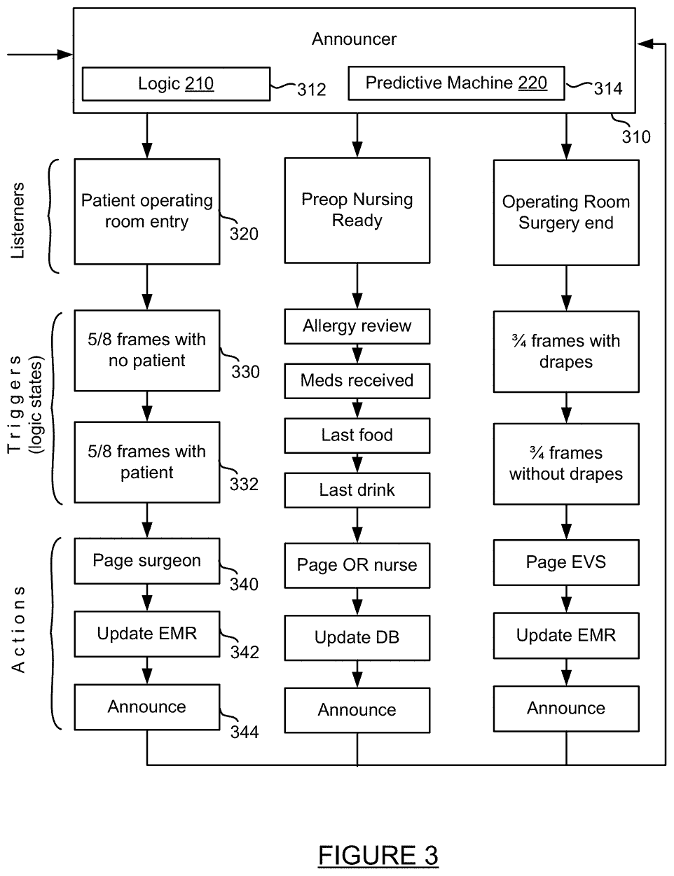

FIG. 3 is a block diagram of an event engine data flow. The event engine of FIG. 3 provides more detail of data flow for event engine 140 of the system of FIG. 1. The event engine 300 of FIG. 3 includes an announcer 310, listeners (including listener 320), triggers (including trigger 330 and 332), and actions (including actions 340, 342, and 344). Announcer 310 may receive data from one or more devices 150-156, from one or more medical facilities. Logic 210 and predictive machine 314 may determine if an event is detected from the received data. Each event is published to the listeners, and each listener compares the events to a requirement or logic for each listener. If a received event satisfies the requirement for a listener, that particular event is forwarded to the appropriate listener by the announcer

A listener receives the object, tracks one or more parameters associated with the object, and optionally performs a result based on those triggers (i.e., logic states). For example, listener 320 associated with “patient operating room entry” receives objects associate with a patient or an entry into an operating room. In particular, if listener 320 determines that five of the last eight frames have no patient (trigger 330), and then five of the next eight frames do have a patient (trigger 332) according, then listener 312 may take one or more actions as a result of the triggers. For example, listener 312 may page a surgeon (action 340), update an Electronic Medical Record (EMR) (action 342), or make an announcement (action 344), for example on a display.

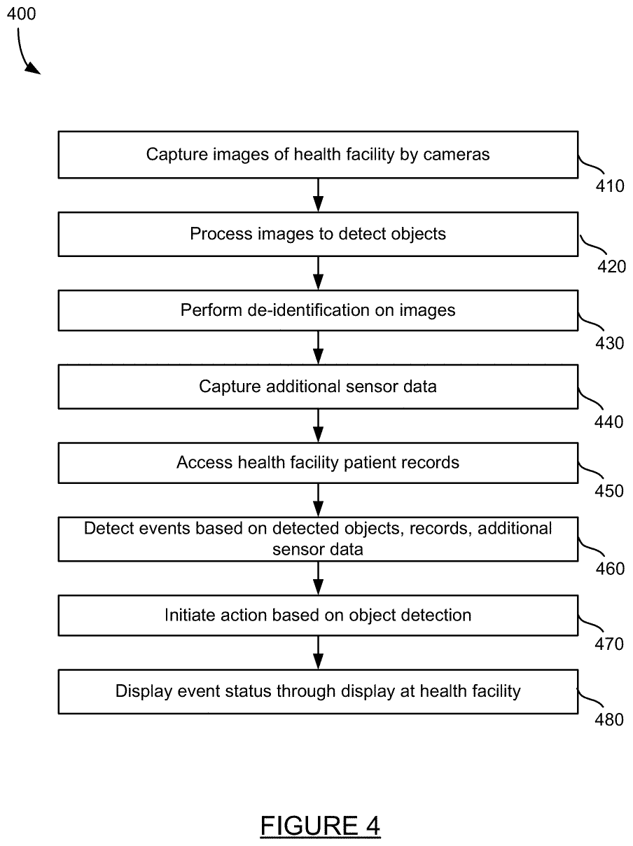

FIG. 4 is an exemplary method for intelligently monitoring a health facility with de-identification of data. FIG. 4 begins with capturing video and/or images of a health facility by one or more cameras. The video and/or images may be captured by room cameras 110 or medical procedure cameras 120. The images are then provided to an image processing application as discussed with respect to FIG. 1.

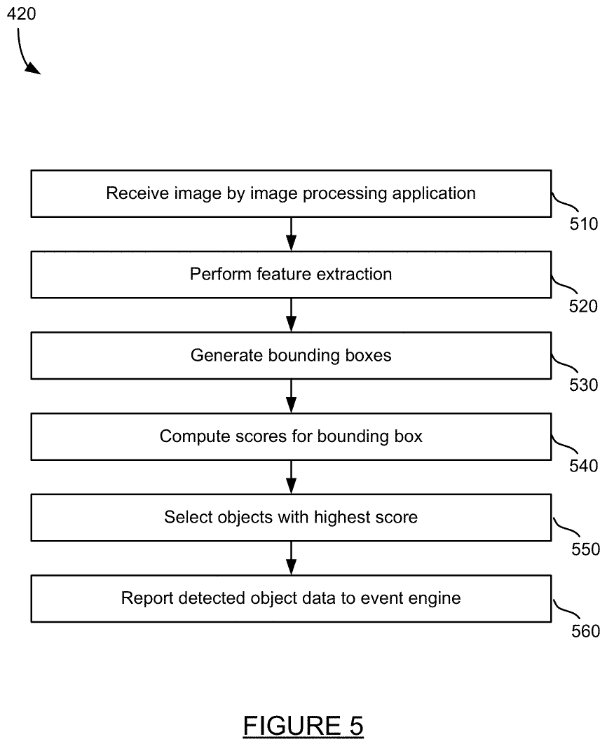

Images are processed to detect objects at step 420. Processing the images may include detecting objects in one or more images associated with a particular time period, such as for example 1, 2, 5, or 10 seconds, or some other time period. Object detection may be performed using several methods, including measuring learning based image processing techniques. More detail for processing images to detect objects is discussed with respect to the method of FIG. 5.

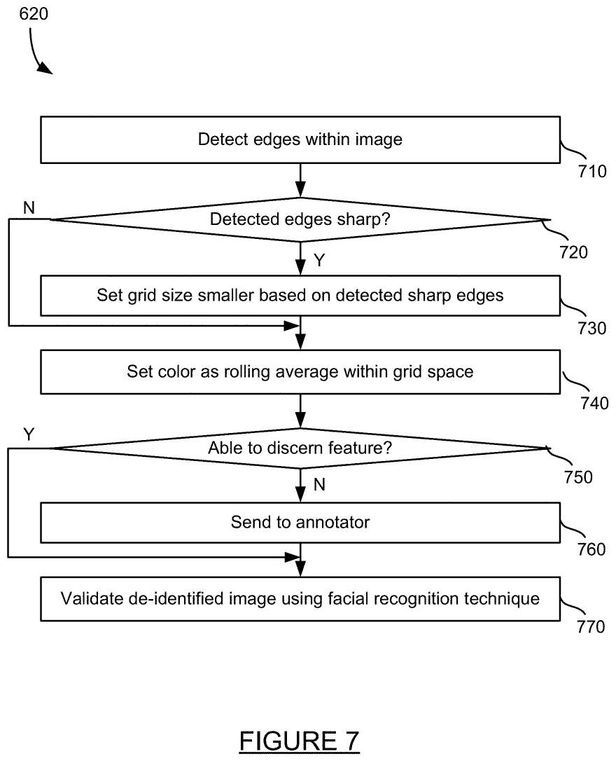

The de-identification is performed on images and optionally other data at step 430. The image de-identification may include blurring images to render any text, labels, badges, and other content unreadable, and modifying pixels within the image to make a person unrecognizable. In some instances, the de-identification may include modifying pixels associated with the user space, head, and/or entire body. Performing the de-identification on images is discussed in more detail below with respect to the method of FIG. 7.

Additional intelligent sensor data may be captured at step 440. The additional sensor data may include data received, by event engine 140, from one or more Bluetooth devices, data received from one or more radio frequency identification (RFID) devices, audio capture devices, and other devices. For example, an RFID tag 152 may be coupled to an object such as a patient bed, and may be read by various RFID readers 153 position throughout a health facility as the patient bed is moved throughout the facility. Event engine 140 may receive intelligent sensor data consisting of an identifier for the RFID tag, RFID reader identifier, timestamp data, and optionally other data.

A Bluetooth device 154 may be coupled to an object such as a patient bed, and may emit a Bluetooth radio frequency signal. The signal transmitted by the Bluetooth device may be broadcast at a specified signal strength. Various beacons 155 may receive the signal as the Bluetooth device is moved around the health facility and determine the strength of the signal at the position of the receiving beacon 155. Event engine 140 may receive intelligent sensor data consisting of the signal strength of the Bluetooth signal at the location of the receiving beacon, diagnostic data such as the Bluetooth device battery level and time in operation, and optionally other data.

An audio may be captured by an audio capture system 156 and provided to event engine 140 as an audio data stream. The audio may be captured by one or more microphones within a particular portion, such as an operating room, of a health facility, it may include one or more talking and/or other audio content. The audio capture system 156 may, in some instances, processor captured audio to remove data that identifies the people talking, as well as the name or other identification information of the patient. In this manner, the audio capture system 156 may perform a de-identification process on the captured audio to provide an audio data stream that is HIPAA compliant.

Health facility patient records are accessed at step 450. In some instances, the health facility patient records may include digital or electronic health data (EHD) for a patient. Accessing patient EHD may include accessing an API associated with the health records database maintained locally by the health facility or remotely by a server. Health facility patient records may be retrieved for a patient undergoing a procedure, seeing a doctor at a particular time, or some other patient record retrieval.

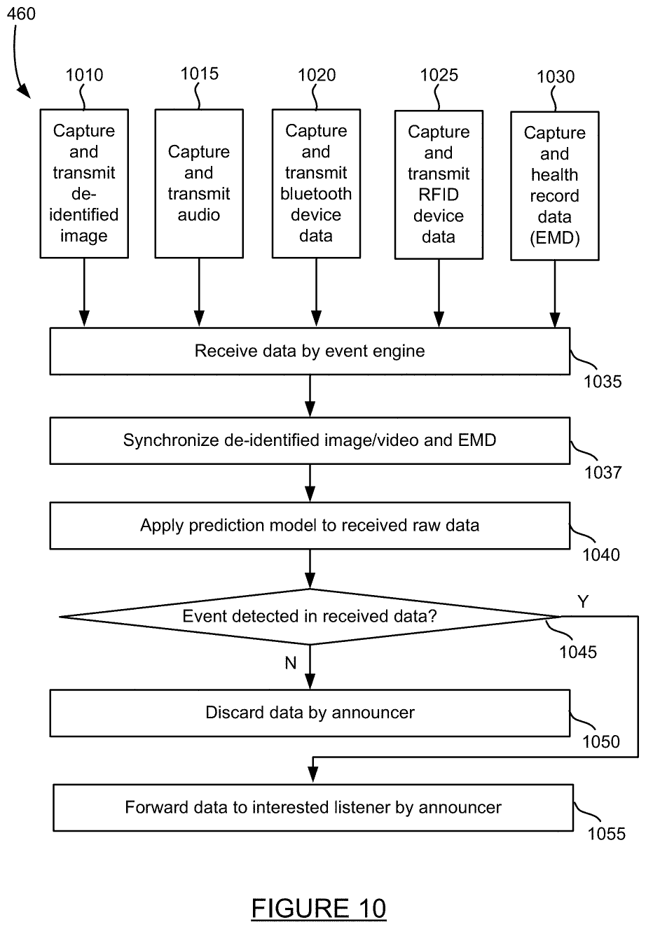

Events are detected based on the object data at step 460. In some instances, after objects are detected in the images, events that correspond to those objects are identified at step 440. Events may be detected by an event engine 140 that receives object data from image processing application 130 as well as data from other intelligent sensors. More detail for detecting events is discussed below with respect to the method of FIG. 10.

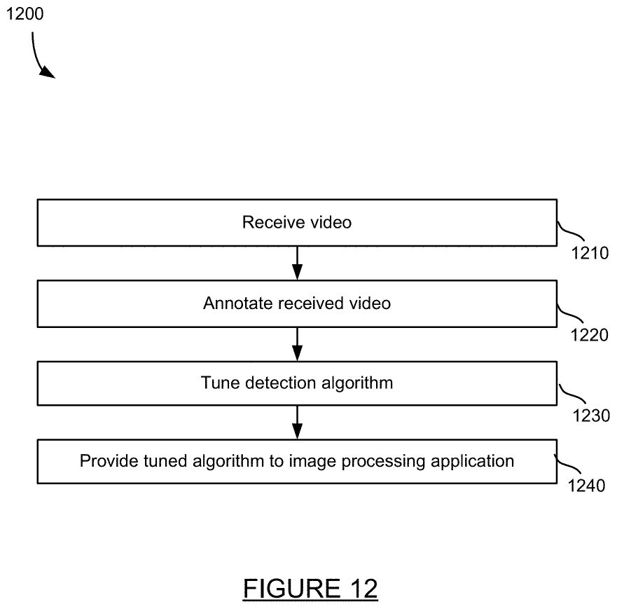

Once events are detected, an action may be initiated based on the object detection at step 470. Initiating actions may include actions taken by one or more listeners that receive object data from an announcer. More detail for initiating action is discussed below with respect to the method of FIG. 12.

Once the actions are initiated, events may be displayed at a health facility at step 480. The output may be provided to an event display 160, cellular device 160, medical records 165, or other devices suitable for communicating information. Event display 160 may be a display located in an operating room, OR prep room, or some other location within a health facility. Event display may output messages, the current state or progress of a procedure, and other information, based on processing by event engine 140. For example, if an anesthesiologist is needed in an operating room, the event engine may detect such and provide a message or update to be displayed on event display 160 regarding the need for the anesthesiologist. Similarly, event information can be transmitted to one or more individual people per their cellular device 160. In some instances, when a procedure is completed, the information can be stored in the patient's medical record, for example if event engine 140 stores data to medical records 165 (which can be the same or different from records managed by records manager 150.

FIG. 5 is an exemplary method for processing images to detect objects. The method of FIG. 5 provides more detail for step 420 the method of FIG. 4. First, an image is received by an image processing application at step 510. Feature extraction is performed on the received image at step 520. The feature extraction may identify features of interest within the image. Bounding boxes are then generated for the extracted features at step 530. Scores may then be computed for each bounding box at step 540. In some instances, data from the feature extraction and bounding boxes is provided to a machine learning based object detection and prediction model. In some instances, the input data is processed by a plurality of machine learning based prediction models, and each model generates a score. Objects within the bounding box are determined to be identified and are selected at step 550. Detected object data is then reported to an event engine at step 560. The object data may include a category of the object, such as “hospital bed” or “patient.”

In some instances, detecting an object within an image can be performed using a machine learning-based technique such as single shot multi-box detector (SSD) 300. Other machine learning-based algorithms, such as YOLO and MASK RCNN, can also be used to detect health facility objects as well. In some instances, the machine learning algorithms can be modified to recognize objects commonly found in a hospital, such as a patient, staff, surgeon, hospital bed, and other health care related workers and devices. For example, video and images may be collected, the video may be annotated to indicate where in the video the object exists, and then the algorithms are trained based on the annotations.

Detected object data is reported to the event engine at step 560. The detected object data may be taken from a label associated with the output of the machine learning-based algorithm. Different machine learning-based algorithms may provide an output of abounding box and label or a perimeter outline of an image with an object label for each object found in the frame. The label may indicate the type of object that was detected. In some instances, a plurality of machine learning algorithms is applied to a frame, each set of algorithms specializing in a particular object. The machine learning algorithm having the highest score, and satisfying a minimum threshold, can be used to determine the type of object. Hence, if a first machine learning algorithm said returns a score of 0.02 for a patient, a second machine learning algorithm said returns the score of 0.06 for a surgeon, and a third machine learning algorithm set returns a score of 0.72 for a patient bed, the object being analyzed is determined to be a patient bed. The label associated with the third machining learning algorithm set, in this case “patient bed,” is reported to the event engine at step 560.

By only sending object information, the raw videos do not leave the health provider room in which they were taken. Rather, processed videos—videos that have undergone a de-identification process—are transmitted to an external device for storage and further processing. Reporting detected object data can include reporting a device identifier or location identifier for the camera which captured the video, a short classification or identifier for the object type, a confidence score for the object detection, and size data for the object. The camera identifier information can be used to determine the location of the camera through a lookup table that pairs cameras identifiers to health facility rooms. The size data may include bounding box information, such as a center point and size of the box, a height, and width of the object.

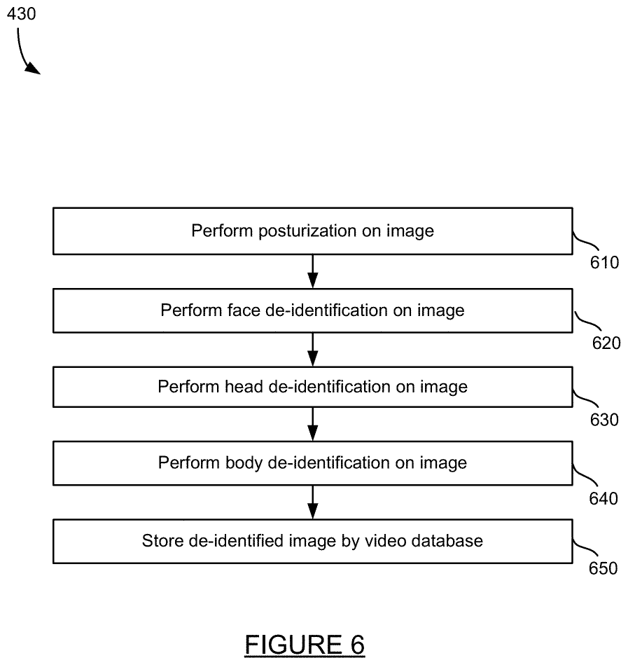

FIG. 6 is a method of performing de-identification on images. The method of FIG. 6 provides more detail for step 430 of the method of FIG. 4. First, posterization is performed on an image at step 610. Posterization of all the pixels in the image renders texts from the image unreadable.

Face de-identification is performed on the image at step 620. Face de-identification includes performing recognition of faces within the image and modifying the pixels of each detected face so that the face cannot be viewed. More detail of an exemplary method for performing de-identification on faces within an image is discussed in more detail with respect to the method of FIG. 7. Head de-identification is performed on the image at step 630. Head de-identification may include modifying pixels that comprise each person's head in the frame. Body de-identification may be performed at step 640. To de-identify a body, a bounding box is generated for each detected body within the frame, and the pixels within the bounding box are modified so that the body cannot be viewed.

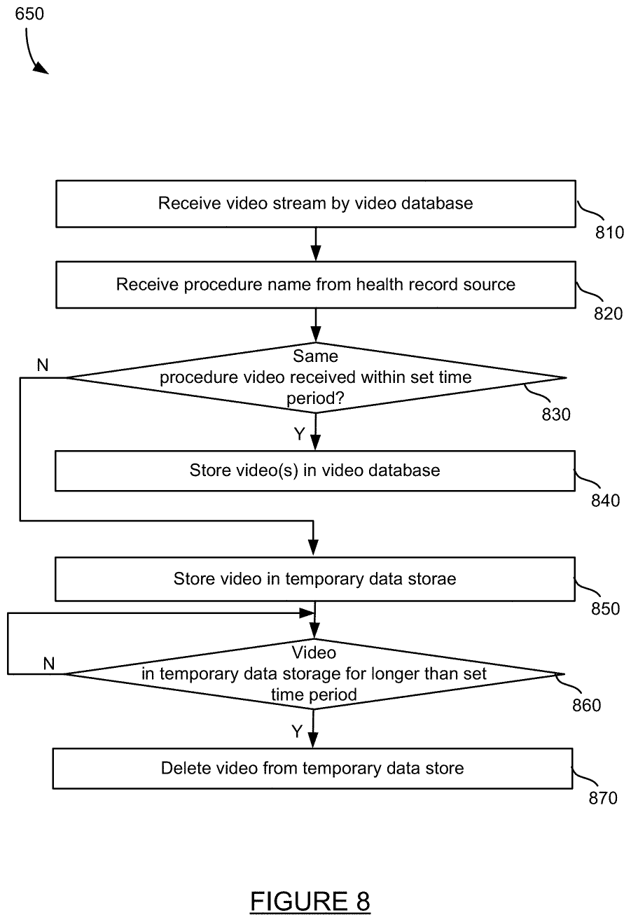

The de-identified image may be transmitted to a video database at step 650. By de-identifying the face, head, and body of every person in the frame, as well as posterizing the image to remove any identification image for any person in the frame, each image has undergone a complete de-identification process so that no patient, health facility worker, or anyone else can be identified in the image. In some instances, videos transmitted to a video database may include only 10% of the original videos taken. By reducing the sampling of videos transmitted to the video database, there is a greater likelihood that people searching for a particular video of a patient will not be able to find it. More detail for transmitting and storing de-identified images is discussed with respect to the method of FIG. 8.

FIG. 7 is a block diagram of a method for performing facial de-identification of images. The method of FIG. 7 provides more detail for step 620 of the method of FIG. 5. First, edges are detected within an image at step 710. A determination is then made as to whether edges within the image are determined to be sharp at step 720. If the edges are not sharp, operation continues to step 740. If the edges are sharp, a grid size for the particular object is set to be smaller than a normal size. A color for the object is determined as a rolling average within the grid space at step 740. A determination is then made as to whether a feature can be discerned from the object at step 750. If a feature can be discerned, then the de-identified object is successful, and a facial recognition technique is performed at step 770. By using the facial recognition technique, a determination can be made as to whether the de-identified images are similar to the non-de-identified image. If the de-identified image and standard image do not match, and the de-identified images are validated at step 770. If a feature is not discernible at step 750, the image is sent to an annotator to discern the particular feature at step 760. After annotating the image, image may be validated at step 770 using a facial recognition technique.

FIG. 8 is a method for storing the identified images in a video database. The method of FIG. 8 provides more detail for step 650 of the method of FIG. 6. First, a video stream is received by video database at step 810. A procedure name for the procedure being performed within the video is received from a health record source at step 820. The procedure name and video may be synchronized with the EMD associated with the patient.

A determination is made as to whether a video for the particular procedure has been received by the video database within a set period of time at step 830. The set period of time may be one week, two weeks, 30 days, two months, or some other time. If another video associated with the same procedure has been received by the video database within the set period of time, the videos are both stored in the video database at step 840. The stored videos have both been processed by a de-identification procedure and are stored with nonidentifying data, such as a procedure name being shown in the video and the name of the hospital at which the procedure took place.

If a video with the same procedure has not been received by the video database within the set time period, the video is stored in a temporary data storage at step 850. If the video is stored in the temporary data storage for longer than the set period of time at step 860, such that no other videos are received by the video database for the same procedure, the video is deleted from the temporary data store at step 870.

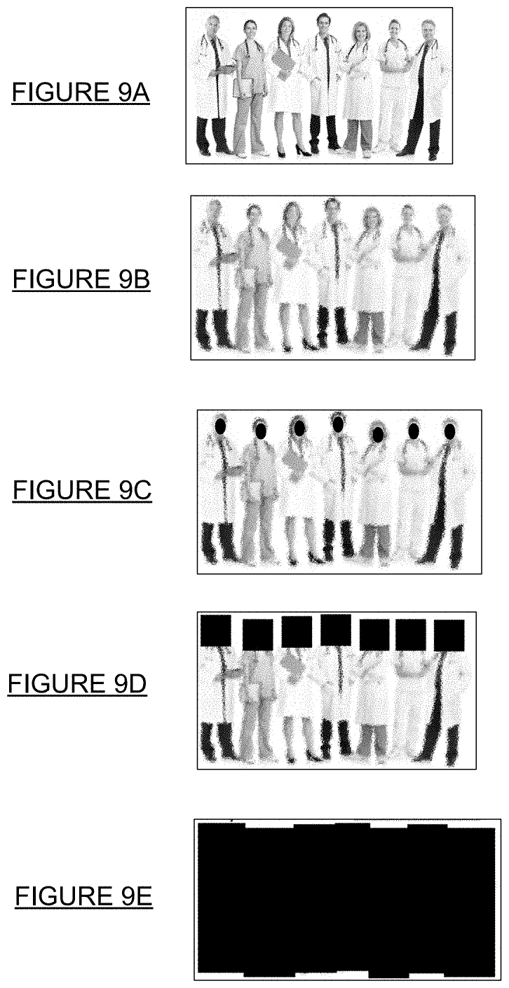

FIGS. 9A-9E illustrated an image going through a de-identification process. The original image illustrated in FIG. 9A depicts the bodies of several health facility workers. The image of FIG. 9A illustrates the workers holding patient charts with their faces de-identified via destruction of the pixels representing their faces. In FIG. 9B, the image of FIG. 9A has been posterized, such that the image is now blurry. As a result, any patient information on a chart, worker name badges, and other text content is illegible and effectively removed.

In FIG. 9C, facial de-identification has been performed on the people in the image. As a result, the pixels comprising the faces of each person have been modified to make their faces unidentifiable. In FIG. 9D, the heads of each person have been de-identified, and pixels forming a bounding box around each person said have been modified to make their head unidentifiable. In FIG. 9E, the bodies of each person have been surrounded with a bounding box, and the pixels of the bounding box have been modified to make the bodies unrecognizable and de-identified.

FIG. 10 is an exemplary method for detecting events based on object data. The method of FIG. 10 provides more detail for step 460 of the method of FIG. 4. First, data is captured and transmitted to an event engine at steps 1010-1030. At step 1010, images are captured from one or more cameras, processed for identification, and the de-identified images as well as object data are transmitted to an event engine. Audio may be captured by one or more audio capture devices at step 1015, the audio undergoes a de-identification process to remove patient and health facility worker identification information, and the processed audio is transferred to the event engine. Bluetooth device data is captured by one or more beacons, a signal strength of the received signal is determined by the beacon (e.g., the level of attenuation of the signal is determined), and the signal strength along with Bluetooth device diagnostic data (battery level of Bluetooth device, time of operation of the Bluetooth device, identifier of the blue tooth device, identification of the beacon) is transmitted to the event engine at step 1020. RFID tags are detected by an RFID reader, and information regarding the identification of the RFID tag, RFID reader, and other data is transmitted to an event engine at step 1025. Health record data (e.g., EHD) is accessed from a health data provider and provided to the event engine at step 1030. The captured and transmitted data is received by an event engine announcer at step 1035.

De-identified video and/or image(s) are synchronized with health record data (i.e., EMD) by the event engine at step 1037. The event engine may synchronize EMD data to the de-identified video, in some instances, as a category for the de-identified video. For example, the EMD may indicate that a patient had laparoscopic gallbladder surgery, and that the surgery ended with a surgical site infection. The event engine may synchronize the de-identified video and EMD data such that the de-identified video is labeled as a gallbladder operation, and that the gallbladder operation ended with a surgical site infection. The labeling does not indicate the date of the gallbladder surgery or any other information that may be used to determine the patient in the de-identified video.

The synchronization can be used to detect potential undesirable outcomes for surgeries or other operations as they occur in real time (e.g., detecting events that match a series of events that occur in previous surgeries with undesired patient outcomes, such as infections). The synchronized de-identified video and EMD can be transmitted to the video data store as discussed with respect to FIG. 8. In some instances, the de-identified video can be labeled or tagged with the synchronized EMD. In some instances, the EMD and de-identified video are transmitted to a video database separately, but the EMD, de-identified video, or both can include reference information to the another.

A prediction model is applied to the received data at step 1040. The prediction model determines whether an event has occurred based on the data received from the intelligent sensors and the health record data at step 1040. The events may include detecting a patient in a room, detecting a patient not in a room, detecting a drink provided to the patient, detecting medications received by patients, detecting an anesthesiologist entering a room, and other events typically occurring in a health facility environment in the course of treating patients and maintaining operation of the health facility.

A determination is made as to whether a prediction module detects an event and receives data at step 1045. In some instances, the prediction model may be implemented by one or more machine learning based algorithms, wherein each set of algorithms is tuned to detect a particular event. Each algorithm set will return a score. The highest score returned by the algorithms may be selected, in some instances as long as higher than a minimum threshold value. The event associated with the set of algorithms having the highest score and above a minimum threshold value may be selected as an event that has occurred. Data may be analyzed to detect events periodically, based on other detected events or based on some other occurrence.

In some instances, an event engine applies the algorithms to the received and health record data (i.e., EMD) to synchronize the data in order to detect events. Hence, the EMD data may indicate that a patient is to have a surgery performed that requires certain steps, and the intelligent sensors generate data that indicates the presence of the patient, patient chart data, and surgeon in an operating room within the health facility. The event engine can synchronize this data to determine the patient associated with the EMD is having the procedure as evidenced by the intelligent sensor data.

If an event is detected based on the received data associated with a particular time, the data is forwarded to an interested listener by the announcer at step 1055. If an event is not detected, the data is discarded by the announcer at step 1050.

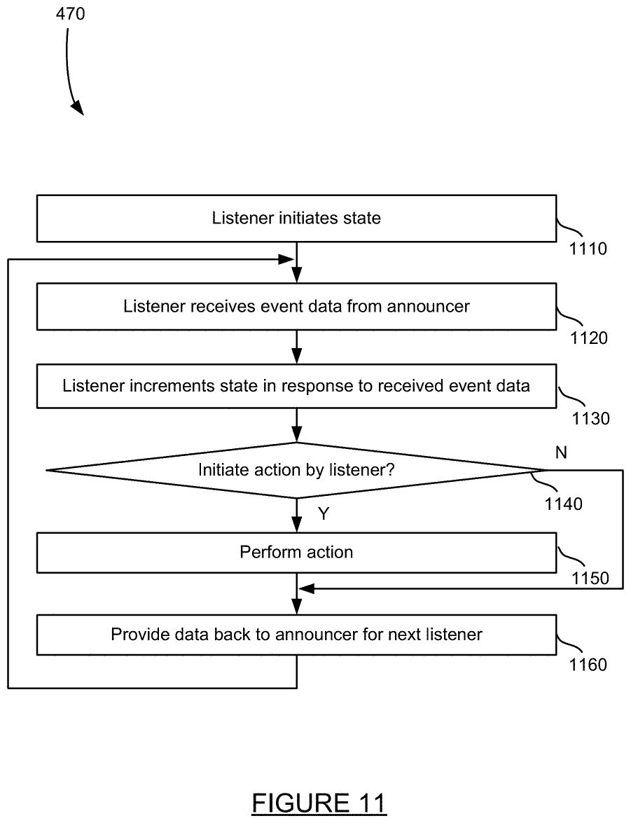

FIG. 11 is a method for initiating action based on an object detection. The method of FIG. 11 provides more detail for step 470 the method of FIG. 4. First, a listener sets an initial state at step 1110. The initial state may be the first state as part of logic implemented by the listener. In some instances, each listener includes logic that watches or “listens” for a series of events. In some instances, the listener may “listen” to receive events from the announcer in a particular order. In some instances, the listener logic does not require all events to be in a particular order—one or more events can be in any order. The listener receives, from an announcer, event data indicating an occurrence of an event at step 1120. If the received event data corresponds to the next event the listener logic is expecting to receive (i.e., the next event the listener is “listening” for), the listener increments a state in response to the received event data at step 1130. If the received event data is not associated with an event the listener is currently listening for, the listener continues to “listen” for events without incrementing a logic state.

After incrementing a state, a determination is made as to whether the listener initiates an action at step 1140. The listener may perform an action if event data has been received to satisfy all the states of the listener logic. If no action should be initiated based on the received event data, the method continues to step 1160 where received event data is provided back to the announcer to be provided to the next listener. If an action should be performed, the action is performed at step 1150, and then the event data is provided to the announcer at step 1160. The method then returns to step 1120 so that the data can be provided to additional listeners, if needed.

FIG. 12 is a method for tuning an intelligent algorithm for processing video. The method of FIG. 12 may be performed on de-identified videos received and stored in video database 170 of FIG. 1. First, video is received at step 1210. Video may be received by video database 170 from one or more image processing applications 130. Received video may have been processed for de-identification of objects such as patients, patient related information, and health facility staff.

The video may be annotated at step 1220. The annotation of the video may be performed by one or more users, or logic implemented by a machine, that draws bounding boxes around items of interest in the video. The items of interest can include patient beds, medications, patients, nurses, surgeons, and other elements that may be relevant when treating a patient or performing an operation on a patient. The items of interest may be annotated, for example, by users. In some instances, video may be annotated in case an object within a room is identified differently by different cameras in the room, or there is some other conflict associated with a particular video.

An algorithm is tuned based on annotation at step 1230. Tuning an algorithm may include applying input into one or more neural networks, or other machine learning based predictive machines or models, in order to tune an algorithm used to detect objects in future images and video. To tune the detection algorithm, the images with the annotated bounding boxes are entered as inputs into the current one or more neural networks. The one or more neural networks process the input and should output images with a bounding box associated with the item of interest. A comparison is then made as to the difference (e.g., error function) between the bounding box generated by annotating the image and the bounding box generated by the one or more neural networks. The neural network of the one or more neural networks that has the most similar bounding box to the annotated bounding box can be determined to be the best neural network, and the algorithm is updated accordingly. For example, the neural network of the one or more neural networks that generated a bounding box which most closely matches the annotated bounding box may receive a larger weighting or otherwise be emphasized within the algorithm. Once the object detection algorithm has been tuned, the tuned (e.g., updated) object detection algorithms may be provided to an image processing application to process subsequent video and images.

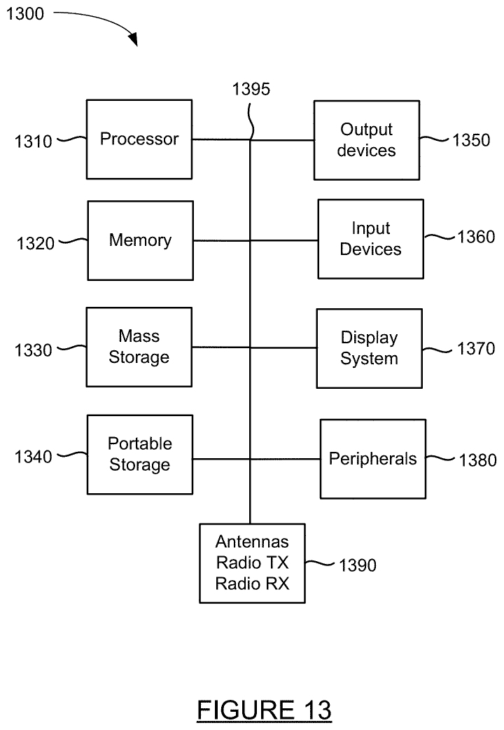

FIG. 13 is an exemplary block diagram of a computing environment 1300. System 1300 of FIG. 13 may be implemented in the contexts of the likes of machines that implement computing machine 132, computing machine 142, video database 170, annotator system 180, algorithm development system 190, event display 160, cellular your device 162, records manager 150, Beacon 155, RFID reader 153, and audio capture system 156 of FIG. 1. The computing system 1300 of FIG. 13 includes one or more processors 1310 and memory 1320. Main memory 1320 stores, in part, instructions and data for execution by processor 1310. Main memory 1320 can store the executable code when in operation. The system 1300 of FIG. 13 further includes a mass storage device 1330, portable storage medium drive(s) 1340, output devices 1350, user input devices 1360, a graphics display 1370, and peripheral devices 1380.

The components shown in FIG. 13 are depicted as being connected via a single bus 1390. However, the components may be connected through one or more data transport means. For example, processor unit 1310 and main memory 1320 may be connected via a local microprocessor bus, and the mass storage device 1330, peripheral device(s) 1380, portable storage device 1340, and display system 1370 may be connected via one or more input/output (I/O) buses.

Mass storage device 1330, which may be implemented with a magnetic disk drive, an optical disk drive, a flash drive, or other device, is a non-volatile storage device for storing data and instructions for use by processor unit 1310. Mass storage device 1330 can store the system software for implementing embodiments of the present invention for purposes of loading that software into main memory 1320.

Portable storage device 1340 operates in conjunction with a portable non-volatile storage medium, such as a floppy disk, compact disk or Digital video disc, USB drive, memory card or stick, or other portable or removable memory, to input and output data and code to and from the computer system 1300 of FIG. 13. The system software for implementing embodiments of the present invention may be stored on such a portable medium and input to the computer system 1300 via the portable storage device 1340.

Input devices 1360 provide a portion of a user interface. Input devices 1360 may include an alpha-numeric keypad, such as a keyboard, for inputting alpha-numeric and other information, a pointing device such as a mouse, a trackball, stylus, cursor direction keys, microphone, touchscreen, accelerometer, and other input devices. Additionally, the system 1300 as shown in FIG. 13 includes output devices 1350. Examples of suitable output devices include speakers, printers, network interfaces, and monitors.

Display system 1370 may include a liquid crystal display (LCD) or other suitable display device. Display system 1370 receives textual and graphical information and processes the information for output to the display device. Display system 1370 may also receive input as a touchscreen.

Peripherals 1380 may include any type of computer support device to add additional functionality to the computer system. For example, peripheral device(s) 1380 may include a modem or a router, printer, and other device.

The system of 1300 may also include, in some implementations, antennas, radio transmitters and radio receivers 1390. The antennas and radios may be implemented in devices such as smart phones, tablets, and other devices that may communicate wirelessly. The one or more antennas may operate at one or more radio frequencies suitable to send and receive data over cellular networks, Wi-Fi networks, commercial device networks such as a Bluetooth device, and other radio frequency networks. The devices may include one or more radio transmitters and receivers for processing signals sent and received using the antennas.

The components contained in the computer system 1300 of FIG. 13 are those typically found in computer systems that may be suitable for use with embodiments of the present invention and are intended to represent a broad category of such computer components that are well known in the art. Thus, the computer system 1300 of FIG. 13 can be a personal computer, handheld computing device, smart phone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Android, as well as languages including Java, .NET, C, C++, Node.JS, and other suitable languages.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto.

1. A system for detecting events in a health facility, comprising:
an image processing application stored and executing on a first computing machine, the image processing machine receiving image data from one or more cameras, the image processing application performing a de-identification process to the image data;
an event engine stored and executing on a second computing machine, the event engine receiving EMD associated with a patient, the event engine synchronizing the de-identified video streams with received electronic medical data; and
a video database that receives the synchronized de-identified video stream and electronic medical data from the event engine,
wherein the stored and synchronized de-identified video stream and electronic medical data is used to update algorithms configured to identify objects in image.

2. The system of claim 1, the event engine receiving sensor data captured by a plurality of sensors physically located within the health facility, the de-identified video stream also synchronized with sensor data.

3. The system of claim 2, wherein the plurality of sensors includes a Bluetooth device.

4. The system of claim 2, wherein the plurality of sensors includes an RFID device.

5. The system of claim 2, wherein the plurality of sensors includes an audio capturing device.

6. The system of claim 1, wherein de-identification includes posterization of the image data and modifying pixels associated with the face and body of a person within an image associated with the image data.

7. The system of claim 1, wherein the computing machine and the one or more cameras are physically positioned within a health facility.

8. The system of claim 7, wherein the de-identified video streams are processed for de-identification before being transmitted to a device outside of the health facility.

9. The system of claim 1, wherein the event engine receives object data from the image processing application, the object data associated with objects detected within the image data by the image processing application

10. The system of claim 1, wherein the health facility is a hospital.

11. The system of claim 1, wherein an algorithm development device updates algorithms configured to identify objects in image data by training and evaluating the updated algorithm.

12. The system of claim 11, wherein the algorithm development device transmits the updated algorithm to the image processing application, the image processing application detecting objects from subsequently received images based on the updated algorithm.

13. A method for detecting events in a health facility, comprising:
receiving image data from one or more cameras by an image processing application stored and executing on a first computing machine, the image processing application performing a de-identification process to the received image data;
receiving electronic medical data associated with a patient by an event engine stored and executing on a second computing machine, the event engine synchronizing the de-identified video streams with received electronic medical data; and
receiving the synchronized de-identified video stream and electronic medical data from the event engine by a video database,
wherein the stored and synchronized de-identified video stream and electronic medical data is used to update algorithms configured to identify objects in image.

14. The method of claim 13, further comprising receiving sensor data by the event engine, the sensor data captured by a plurality of sensors physically located within the health facility, the de-identified video stream also synchronized with sensor data.

15. The system of claim 14, wherein the plurality of sensors includes a Bluetooth device.

16. The system of claim 14, wherein the plurality of sensors includes an RFID device.

17. The system of claim 14, wherein the plurality of sensors includes an audio capturing device.

18. The system of claim 13, wherein the de-identification process includes posterization of the image data and modifying pixels associated with the face and body of a person within an image associated with the image data.

19. The system of claim 13, wherein the computing machine and the one or more cameras are physically positioned within a health facility,

20. The system of claim 19, wherein the de-identified video streams are processed for de-identification before being transmitted to a device outside of the health facility.

21. The system of claim 13, further comprising receiving object data from the image processing application by the event engine, the object data associated with objects detected within the image data by the image processing application.

22. The system of claim 13, wherein the health facility is a hospital.

23. The system of claim 13, wherein an algorithm development device updates algorithms configured to identify objects in image data by training and evaluating the updated algorithm.

24. The system of claim 23, further comprising transmitting, by the algorithm development device, the updated algorithm to the image processing application, the image processing application detecting objects from subsequently received images based on the updated algorithm.
